(19) United States
(12) Patent Application Publication
(10) Pub. No.: US 2012/0190100 A1
(43) Pub. Date: Jul. 26, 2012

(54) ENZYMATIC COMPOSITION FOR THE DIGESTION OF CHICKEN EMBRYOS

(75) Inventors: Martine Marigliano, Schiltigheim (FR); Jacqueline Reymund, Reitwiller (FR); Martine Sainte-Marie, Nothalten (FR)

(73) Assignee: Transgene AS, lllkirch (FR)

(21) Appl. No.: 13/386,168

(22) PCT Filed: Jul. 21, 2010
(86) PCT No.: PCT/EP2010/004478

§ 371 Date: Apr. 13, 2012

Related U.S. Application Data
(60) Provisional application No. 61/264,299, filed on Nov. 25, 2009.

(30) Foreign Application Priority Data
Jul. 21, 2009 (EP) 09305690.1

(51) Int. Cl. C12N 7/02; C12S 3/00

(52) U.S. Cl. 435/239; 435/267

(57) ABSTRACT

The present invention relates to an enzymatic composition for the digestion of chicken embryos intended to the preparation of cells which are used for the production of viruses. The present invention also relates to a method for producing a wild type, an attenuated and/or a recombinant virus comprising a step of preparation of cells from chicken embryos using an enzymatic composition of the invention. The present invention relates to a purified wild type, attenuated and/or recombinant virus obtained and to a pharmaceutical composition, preferably a vaccine, comprising said virus for the treatment and/or the prevention a cancer, an infectious disease and/or an autoimmune disorder, and uses thereof.

### TECHNICAL FIELD

The present invention pertains to the field of virus production. In particular, the invention relates to an enzymatic composition for the digestion of chicken embryos intended to the preparation of cells which are used for the production of viruses.

### BACKGROUND OF THE INVENTION

Most viral vaccines such as attenuated or recombinant viruses are manufactured from primary or secondary chicken cells such as for instance chicken embryo fibroblasts (CEFs), chicken embryo kidney cells (CEKCs) or chicken embryo liver cells (CELCs). Primary CEFs are notably used for the production of japanese encephalitis virus vaccines (manufactured by e.g. Pasteur Merieux), yellow fever virus vaccines (manufactured by e.g. Arilvax), influenza virus vaccines (manufactured by e.g. Medeva Pharmaceuticals), measles and rubella virus vaccines (manufactured by e.g. Merck) and modified Vaccinia virus Ankara (MVA) vaccines.

Several methods have been developed to generate cell preparations from primary tissues. These methods involve the use of mechanical dissociation, enzymatic dissociation or a combination of both. Mechanical dissociation consists for instance of scraping the embryos with a scalpel, mincing the embryos or physically cutting the embryos apart. Excessive mechanical dissociation involve often result in a significant amount of cell death and cell damage. Moreover the manual nature of certain mechanical dissociation protocols (e.g. trituration, which is done by hand) often make it difficult compare measured values (such as cell viability) from different sources since dissociation efficiency varies between individuals. In fact, the manual nature of this procedure may contribute to differences in the physical attributes (e.g. cell concentration, cell viability, cell size distribution) between two otherwise identical samples. In an attempt to avoid the negative consequences of mechanical dissociation, trypsin from bovine pancreas was used. Unfortunately, trypsin from bovine pancreas may contain pathogenic agents such as viruses. There was therefore a potential risk that these pathogenic agents are transmitted to the animals or humans to be treated or vaccinated with the vaccine. One of the many potential major problems associated with the commonly used trypsin from bovine pancreas is the possibility to transmit the agent causing bovine spongiforme encephalopathy (BSE) to the animals or humans that come into contact with the products produced from cell culture. As a consequence recombinant trypsin have been developed for the production of vaccines from primary or secondary cells.

International patent application WO 2004/022729 describes a method for the amplification of a poxvirus on primary CEFs, wherein said CEFs are obtained from chicken embryos treated with a prewarmed (37° C.) trypsin-EDTA solution for 15 minutes in presence of serum free medium at room temperature.

International patent application WO 2006/116803 describes a method for producing stem cells from mammalian embryos wherein the embryos may be immersed in a pre-warmed trypsin solution for between 5 to 60 minutes depending on the size of the embryos.

One major problem for the production of vaccines from primary chicken cells is the provision of a sufficient amount of embryos. In order to reduce the number of embryos required, enzymatic compositions leading to the obtention of cell preparations consisting in a maximal number of cells extracted per embryo are needed. The present invention provides such compositions. The present invention provides more particularly enzymatic compositions for the digestion of chicken embryos leading to the obtention of cell preparations consisting in more than 500 10⁶ cells extracted per embryo. The enzymatic compositions of the invention are moreover able to digest chicken embryos which have not been previously dissected.

### DISCLOSURE OF THE INVENTION

As used throughout the entire application, “a” and “an” are used in the sense that they mean “at least one”, “at least a first”, “one or more” or “a plurality” of the referenced components or steps, unless the context clearly dictates otherwise.

As used throughout the entire application, “and/or” wherever used herein includes the meaning of “and”, “or” and “all or any other combination of the elements connected by said term”.

As used throughout the entire application, “comprising” and “comprise” are intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used throughout the entire application, “about” or “approximately” as used herein means within 20%, preferably within 10%, and more preferably within 5% of a given value or range.

The present invention relates to an enzymatic composition for the digestion of chicken embryos intended to the preparation of cells. According to one special embodiment, said cells can be used for the production of viruses.

The present invention relates more particularly to the use of an enzymatic composition for the digestion of chicken embryos leading to the obtention of a chicken cell preparation.

The enzymatic composition of the invention surprisingly leads to the obtention of a chicken cell preparation consisting of more than 500 10⁶ cells extracted per embryo. With this regard, the present invention relates more particularly to an enzymatic composition for the digestion of chicken embryos leading to the obtention of a chicken cell preparation consisting of more than 500 10⁶ cells extracted per embryo.

The enzymatic composition of the invention comprises at least 2 enzymes. The enzymes comprised in the composition of the invention are selected from the group consisting of trypsin, chymotrypsin, trypsinogen, chymotrypsinogen, dispase, collagenase, accutase, thermolysin, pronase, hyaluronidase, elastase, papain, neuraminidase and pancreatin. The enzymes comprised in the composition of the invention are preferably selected from the group consisting of trypsin, dispase, collagenase and accutase. With this regard, the present invention relates more particularly to an enzymatic composition for the digestion of chicken embryos, wherein said enzymatic composition comprises at least 2 enzymes selected from the group consisting of trypsin, dispase, collagenase and accutase.

According to a preferred embodiment of the invention, the enzymatic composition of the invention is selected from the group consisting of:
trypsin, dispase and collagenase;
trypsin, dispase and accutase;
trypsin, dispase, collagenase and accutase;
trypsin and dispase; and
dispase and accutase.

According to one special embodiment of the invention, the enzymatic composition of the invention is selected from the group consisting of:
trypsin added at a concentration equivalent to 30 mL/Embryo of TrypLE Select from Invitrogen Cat. No. 12563-029, 10 mL/Embryo of dispase 10 mg/mL, and 10 mL/Embryo of collagenase 20 mg/mL;
trypsin added at a concentration equivalent to 10 mL/Embryo of TrypLE Select from Invitrogen Cat. No. 12563-029, 10 mL/Embryo of dispase 10 mg/mL, and 10 mL/Embryo of collagenase 20 mg/mL;
trypsin added at a concentration equivalent to 15 mL/Embryo of TrypLE Select from Invitrogen Cat. No. 12563-029, 10 mL/Embryo of dispase 10 mg/mL, and accutase added at a concentration equivalent to 10 mL/Embryo of Accutase from Sigma Cat. No. A-6964;
trypsin added at a concentration equivalent to 15 mL/Embryo of TrypLE Select from Invitrogen Cat. No. 12563-029, 10 mL/Embryo of dispase 10 mg/mL, 10 mL/Embryo of collagenase 20 mg/mL, and accutase added at a concentration equivalent to 10 mL/Embryo of Accutase from Sigma Cat. No. A-6964;
trypsin added at a concentration equivalent to 30 mL/Embryo of TrypLE Select from Invitrogen Cat. No. 12563-029, 10 mL/Embryo of dispase 10 mg/mL, and accutase added at a concentration equivalent to 10 mL/Embryo of Accutase from Sigma Cat. No. A-6964;
trypsin added at a concentration equivalent to 10 mL/Embryo of TrypLE Select from Invitrogen Cat. No. 12563-029, 10 mL/Embryo of dispase 10 mg/mL, and 5 mL/Embryo of collagenase 20 mg/mL;
trypsin added at a concentration equivalent to 30 mL/Embryo of TrypLE Select from Invitrogen Cat. No. 12563-029, 10 mL/Embryo of dispase 10 mg/mL, 10 mL/Embryo of collagenase 20 mg/mL, and accutase added at a concentration equivalent to 10 mL/Embryo of Accutase from Sigma Cat. No. A-6964;
trypsin added at a concentration equivalent to 10 mL/Embryo of TrypLE Select from Invitrogen Cat. No. 12563-029, 10 mL/Embryo of dispase 10 mg/mL, and accutase added at a concentration equivalent to 10 mL/Embryo of Accutase from Sigma Cat. No. A-6964;
trypsin added at a concentration equivalent to 15 mL/Embryo of TrypLE Select from Invitrogen Cat. No. 12563-029, and 5 mL/Embryo of dispase 10 mg/mL;
trypsin added at a concentration equivalent to 30 mL/Embryo of TrypLE Select from Invitrogen Cat. No. 12563-029, and 10 mL/Embryo of dispase 10 mg/mL;
trypsin added at a concentration equivalent to 10 mL/Embryo of TrypLE Select from Invitrogen Cat. No. 12563-029, and 10 mL/Embryo of dispase 10 mg/mL;
10 mL/Embryo of dispase 10 mg/mL, and accutase 10 mL/Embryo; and
trypsin added at a concentration equivalent to 5 mL/Embryo of TrypLE Select from Invitrogen Cat. No. 12563-029, and 15 mL/Embryo of dispase 10 mg/mL.

According to one more special embodiment of the invention, the enzymatic composition of the invention is selected from the group consisting of:
trypsin added at a concentration equivalent to 30 mL/Embryo of TrypLE™ Select from Invitrogen Cat. No. 12563-029, 10 mL/Embryo of dispase 10 mg/mL from Invitrogen Cat. No. 17105-041, and 10 mL/Embryo of collagenase III 20 mg/mL from Gibco Invitrogen Cat No. 17102;
trypsin added at a concentration equivalent to 10 mL/Embryo of TrypLE™ Select from Invitrogen Cat. No. 12563-029, 10 mL/Embryo of dispase 10 mg/mL from Invitrogen Cat. No. 17105-041, and 10 mL/Embryo of collagenase III 20 mg/mL from Gibco Invitrogen Cat No. 17102;
trypsin added at a concentration equivalent to 15 mL/Embryo of TrypLE™ Select from Invitrogen Cat. No. 12563-029, 10 mL/Embryo of dispase 10 mg/mL from Invitrogen Cat. No. 17105-041, and accutase added at a concentration equivalent to 10 mL/Embryo of Accutase™ from Sigma Cat. No. A-6964;
trypsin added at a concentration equivalent to 15 mL/Embryo of TrypLE™ Select from Invitrogen Cat. No. 12563-029, 10 mL/Embryo of dispase 10 mg/mL from Invitrogen Cat. No. 17105-041, 10 mL/Embryo of collagenase III 20 mg/mL from Gibco Invitrogen Cat No. 17102, and accutase added at a concentration equivalent to 10 mL/Embryo of Accutase™ from Sigma Cat. No. A-6964;
trypsin added at a concentration equivalent to 30 mL/Embryo of TrypLE™ Select from Invitrogen Cat. No. 12563-029, 10 mL/Embryo of dispase 10 mg/mL from Invitrogen Cat. No. 17105-041, and accutase added at a concentration equivalent to 10 mL/Embryo of Accutase™ from Sigma Cat. No. A-6964;
trypsin added at a concentration equivalent to 10 mL/Embryo of TrypLE™ Select from Invitrogen Cat. No. 12563-029, 10 mL/Embryo of dispase 10 mg/mL from Invitrogen Cat. No. 17105-041, and 5 mL/Embryo of collagenase III 20 mg/mL from Gibco Invitrogen Cat No. 17102;
trypsin added at a concentration equivalent to 30 mL/Embryo of TrypLE™ Select from Invitrogen Cat. No. 12563-029, 10 mL/Embryo of dispase 10 mg/mL from Invitrogen Cat. No. 17105-041, 10 mL/Embryo of collagenase III 20 mg/mL from Gibco Invitrogen Cat No. 17102, and accutase added at a concentration equivalent to 10 mL/Embryo of Accutase™ from Sigma Cat. No. A-6964;
trypsin added at a concentration equivalent to 10 mL/Embryo of TrypLE™ Select from Invitrogen Cat. No. 12563-029, 10 mL/Embryo of dispase 10 mg/mL from Invitrogen Cat. No. 17105-041, and accutase added at a concentration equivalent to 10 mL/Embryo of Accutase™ from Sigma Cat. No. A-6964;
trypsin added at a concentration equivalent to 15 mL/Embryo of TrypLE™ Select from Invitrogen Cat. No. 12563-029, and 5 mL/Embryo of dispase 10 mg/mL from Invitrogen Cat. No. 17105-041;
trypsin added at a concentration equivalent to 30 mL/Embryo of TrypLE™ Select from Invitrogen Cat. No. 12563-029, and 10 mL/Embryo of dispase 10 mg/mL from Invitrogen Cat. No. 17105-041;
trypsin added at a concentration equivalent to 10 mL/Embryo of TrypLE™ Select from Invitrogen Cat. No. 12563-029, and 10 mL/Embryo of dispase 10 mg/mL from Invitrogen Cat. No. 17105-041;
10 mL/Embryo of dispase 10 mg/mL from Invitrogen Cat. No. 17105-041, and accutase 10 mL/Embryo; and
trypsin added at a concentration equivalent to 5 mL/Embryo of TrypLE™ Select from Invitrogen Cat. No. 12563-029, and 15 mL/Embryo of dispase 10 mg/mL from Invitrogen Cat. No. 17105-041.

According to the invention, the enzymatic composition is free from animal products. As used throughout the entire application, <<animal products>> refer to any compound or collection of compounds that was produced in or by an animal cell in a living organism. With this regard, the enzymes comprised in the composition of the invention are recombinant enzymes.

Trypsin is a serine protease produced in the pancreas of many vertebrates. Trypsin catalyses a hydrolytic cleavage of peptides at the carboxyl group of the basic amino acids arginine and lysine. Many recombinant trypsin have been already described such as e.g. recombinant trypsin from Pishia pastoris (Roche Applied Science, September 2003) and some of them are commercially available such as e.g. TrypLE™ Select (Invitrogen, Cat. No. e.g. 12563-011; 12563-029) or TrypZean (Sigma, Cat. No. e.g. T-3449). Preferred recombinant trypsin used according to the invention is TrypLE™ Select (Invitrogen, Cat. No. 12563-029) as described in the examples.

Dispase is a neutral protease produced from Bacillus polymyxa bacteria (Dispase, a neutral protease from Bacillus polymyxa, is a powerful fibronectinase and type IV collagenase, STENN K. S., LINK R., MOELLMANN G., MADRI J., KUKLINSKA E., J. Invest Dermatol. 1989 August; 93(2):287-90). Many recombinant dispase have been already described such as e.g. recombinant dispase I, recombinant dispase II (Roche Applied Science, August 2004) or BD Dispase (BD Biosciences), and some of them are commercially available such as e.g. Dispase (Invitrogen, Cat. No. e.g. 17105-041), Dispase I (Sigma, Cat. No. e.g. D4818) or Dispase (StemCell Technologies, Cat. No. e.g. 07913; 07923). Preferred recombinant dispase used according to the invention is Dispase (Invitrogen, Cat. No. 17105-041) as described in the examples.

Collagenase is a protease produced from Clostridium histolyticum bacteria that breaks the peptide bonds in collagen (Isolation and characterization of proteinase and collagenase from Cl. Histolyticum, MANDL I., MACLENNAN J. D., HOWES E. L., J Clin Invest. 1953 December; 32(12):1323-9). Many collagenase have been already described such as e.g. recombinant collagenase III (Collagenase III: A superior enzyme for complete disaggregation and improved viability of normal and malignant human breast tissue, SPEIRS V., WHITE M. C. and GREEN A. R., In vitro Cell. Dev. Biol.-Animal, 32:72-74, February 1996), and some recombinant collagenase are commercially available such as e.g. Collagenase type I (Sigma, Cat. No. e.g. C0130; C1639), Collagenase type II (Sigma, Cat. No. e.g. C0130; C1764; C6885), Collagenase type III (e.g. Gibco Invitrogen, Cat. No. e.g. 17102; Sigma, Cat. No. e.g. C0255), Collagenase type IV (Sigma, Cat. No. e.g. C1889), Collagenase type V (Sigma, Cat. No. e.g. C2014; C9263), Collagenase type VII (Sigma, Cat. No. e.g. C0773; C2399) or Collagenase type XI (Sigma, Cat. No. e.g. C4785; C7657). Preferred recombinant collagenase used according to the invention is collagenase III (Gibco Invitrogen Cat. No. 17102) as described in the examples.

Accutase is a protease of crustacean origin. Recombinant accutase are commercially available such as e.g. Accutase (PAA, Cat. No. e.g. L11-007), Accutase (Thermo, Cat. No. e.g. 21-201-0100V), Accutase (Interchim, Cat. No. e.g. UPN68081), Accutase (Sigma, Cat. No. e.g. A6964), Accutase (eBioscience, Cat. No. e.g. 00-4555) or Accutase (Millipore, Cat. No. e.g. SCR005). Preferred recombinant accutase used according to the invention is Accutase (Sigma, Cat. No. A6964) as described in the examples.

The “cells” prepared by digestion of chicken embryos with the enzymatic compositions of the invention include but are not limited to fibroblasts, kidney cells, liver cells, cardiac cells, muscle cells, epithelial cells, blood cells and endothelial cells.

According to one special embodiment of the invention, the enzymatic composition of the invention is used for the digestion of chicken embryos leading to the obtention of a chicken cell preparation consisting of isolated chicken cells. According to the invention, said isolated chicken cells are chicken embryo fibroblasts (CEFs), chicken embryo kidney cells (CEKCs) or chicken embryo liver cells (CELCs), and preferably chicken embryo fibroblasts (CEFs).

According to another special embodiment of the invention, the enzymatic composition of the invention is used for the digestion of chicken embryos leading to the obtention of a chicken cell preparation consisting of a mixture of chicken cells. According to the invention, said mixture of chicken cells comprises CEFs, CEKCs, CELCs, cardiac cells, muscle cells, epithelial cells, blood cells and/or endothelial cells.

The cells are preferably extracted from Specific Pathogen Free (SPF) eggs. SPF eggs are commercially available e.g. from Charles River Laboratories (Wilmington, Mass., USA). Said eggs are preferably more than 9 days old, more preferably between 10 and 14 days old and even more preferably are 12 days old. Before the extraction of the embryos, eggs are preferably disinfected. Many methods and products dedicated to the disinfection of eggs are available in the prior art. Incubation in a formol solution (e.g. 2% formol during e.g. 1 minute) followed by a rinsing in ethanol (e.g. 70% ethanol) is particularly preferred. Then the eggs are opened, the embryos are extracted, and heads and feet are cut off.

The embryos, not dissected, are then directly digested by the enzymatic composition of the invention. According to the invention, the cell disaggregation is obtained under the following conditions:
a temperature of incubation comprised between 35° C. and 39° C., preferably between 36° C. and 37° C., more preferably of 36° C., 36.5° C. or 37° C., and even more preferably of 37° C.; and
a duration of incubation comprised between 1 and 3 hours, and preferably of 2 hours.

The enzymatic compositions of the invention are surprisingly able to digest chicken embryos which have not been previously dissected.

According to specific embodiment of the invention, the enzymes of the enzymatic composition can also be added to the embryos at different times. For instance, for an enzymatic composition comprising trypsin, dispase and collagenase, the embryos can first be digested in presence of dispase and collagenase and then, after a time interval, digested in presence of trypsin. As an other example, for an enzymatic composition comprising trypsin, dispase and accutase, the embryos can first be digested in presence of trypsin, then after a time interval, digested in presence of dispase, and finally after a time interval (which can be the same or different from the previous time interval) digested in presence of accutase. According to the invention, said time interval is comprised between 15 minutes and 90 minutes, preferably between 20 and 80 minutes, more preferably between 30 and 60 minutes, and is more preferably 30 minutes, 35 minutes, 40 minutes, 45 minutes, 50 minutes, 55 minutes or 60 minutes, and is even more preferably 60 minutes.

The mixture obtained is then filtrated (e.g. using a seave made of inox commercially available e.g. from Fischer Bioblock, Cat No. A37532) to remove the undigested tissues. The cells (e.g. CEFs; CEKCs; CELCs) are then collected by centrifugation (e.g. 2300 rpm, 15 minutes). Said centrifugation also permits to remove the enzymes (i.e. enzymes will be in the supernatant). Method and conditions as described in Example 1 are preferably used.

According to the invention, the primary cells obtained can also either be used directly or after one further cell passage as secondary cells.

The cells (i.e. primary or secondary) are then cultivated in an appropriate cell culture medium. Cell culture media used according to the invention are preferably free from animal product. Many media free from animal product has been already described and some of them are commercially available such as for instance 293 SFM II; 293-F Cells, SFM Adapted; 293-H Cells, SFM Adapted; 293Fectin™ Transfection Reagent; CD 293 AGT™; CD 293 Medium; FreeStyle™ 293 Expression System; FreeStyle™ 293 Medium; FreeStyle™ 293-F Cells, SFM Adapted; VP-SFM; VP-SFM ACT™; Adenovirus Expression Medium (AEM) Growth Medium for PER.C6® Cells; CD 293 ACT™; CD 293 Medium; COS-7L Cells, SFM Adapted; EPISERF® Medium; OptiPro™ SFM (all available from Invitrogen). The cells are preferably cultivated between 1 and 5 days, more preferably between 1 and 2 days and even more preferably for 2 days before infection. The cells are preferably cultivated at a temperature comprised between 30 and 37° C.

The present invention also relates to a method for producing a wild type, an attenuated and/or a recombinant virus comprising a step of preparation of cells from chicken embryos using an enzymatic composition as previously described.

The method for producing a wild type, an attenuated and/or a recombinant virus comprising a step of preparation of cells from chicken embryo using an enzymatic composition of the invention, may also comprise a step of infection of said cells with a virus. Step of infection cells with a virus is well known to the one skilled in the art. As used throughout the entire application, “infection” refers to the transfer of the viral nucleic acid to a cell, wherein the viral nucleic acid is replicated, viral proteins are synthesized, or new viral particles assembled. The one skilled in the art is able to select the most appropriate cells for the production of a specific virus. For instance, in the specific embodiment where the virus to produce is a poxvirus, the cells used are preferably CEFs. Step of infection of cells with a virus is performed in an appropriate cell culture medium which can be the same or different from the cell culture medium used for the preparation of said cells. Cell culture media used according to the invention are preferably free from animal product. Many media free from animal product have been already described and some of them are commercially available as previously described. Preferred cell culture, media used for the infection step is Basal Medium Eagle cell culture medium (Invitrogen). The cell culture medium is preferably seeded with between 0.5 to 1.5 and more preferably between 1.1 and 1.3 and even more preferably about 1.2 Embryo/L of cell culture medium. In the specific embodiment where the virus to produce is MVA, the MVA is seeded in the cell culture vessel at a MOI which is preferably comprised between 0.001 and 0.1, more preferably between 0.03 and 0.07 and even more preferably about 0.05.

The method for producing a wild type, an attenuated and/or a recombinant virus comprising a step of preparation of cells from chicken embryo using an enzymatic composition of the invention, may also comprise a step of culture of the infected cells. Step of culture of the infected cells until progeny virus is produced is well known to the one skilled in the art. Said step can comprise growth adhering to surfaces, growth in suspension in presence or not of (micro)carriers, or combinations thereof. Culturing can be done for instance in dishes, roller bottles or in bioreactors, using batch, fed-batch, continuous systems, hollow fiber, and the like. In order to achieve large scale production of virus through cell culture it is preferred in the art to have cells capable of growing in suspension in presence or not of (micro)carriers, and it is preferred to have cells capable of being cultured in media free from animal product. Cell culture media used according to the invention are preferably free from animal product. Many media free from animal product have been already described and some of them are commercially available as previously described. Step of culture of the infected cells is performed in an appropriate cell culture medium which can be the same or different from the cell culture medium used for the preparation of said cells and from the cell culture medium used for the infection of said cells with a virus. Preferred cell culture medium used for the culture of infected cells is Basal Medium Eagle (Invitrogen). The infected cells are preferably cultivated for between 1 and 6 days, more preferably between 2 and 4 days and even more preferably 3 days. The infected cells are preferably cultivated at a temperature comprised between 30° C. and 37° C.

The method for producing a wild type, an attenuated and/or a recombinant virus comprising a step of preparation of cells from chicken embryos using an enzymatic composition of the invention, may also comprise a step of recovering of the viruses produced from the supernatant and/or from the cells. When the viruses are recovered from the cells (i.e. from the cells only, or from the cells and from the supernatant), the step of recovering of the viruses produced can be preceded by a step allowing the disruption of the cell membrane. This step leads to the liberation of the viruses from the cells. The disruption of the cell membrane can be induced by various techniques well known by the one skilled in the art. These techniques comprise but are not limited to freeze/thaw, hypotonic lysis, sonication (by using a sonicator) and microfluidization (by using a microfluidizer). Sonicators are commercially available from e.g. Heraeus PSP, Biologics, Misonix or GlenMills. Preferred sonicators used according to the present invention are SONITUBE 20 kHz type SM 20-120-3 and SONITUBE 35 kHz type SM 35-400-3 (Heraeus PSP). Microfluidizers are commercially available from e.g. Microfluidics Corporation. The packaging cell membrane can also be disrupted by using a using a SLM Aminco French press. The packaging cell membrane can also be disrupted by using a high speed homogenizer. High speed homogenizers are commercially available from e.g. Silverson Machines or Ika-Labotechnik. Preferred high speed homogeneizer used according to the present invention is a SILVERSON L4R (Silverson Machines).

The method for producing a wild type, an attenuated and/or a recombinant virus comprising a step of preparation of cells from chicken embryos using an enzymatic composition of the invention, may also comprise one or more steps of purification of the recovered viruses. Purification step(s) can be for instance but are not limited to:
A clarification allowing under suitable conditions the withdrawal of the cellular debris. Said clarification can be performed by e.g. depth filtration. Depth filtration includes but is not limited to the use of one or more commercially available products such as Sartopure® filters from Sartorius (e.g. Sartopure® PP2), CUNO Incorporated AP series depth filters (e.g. AP01), CUNO Incorporated CP series depth filters (e.g. CP10, CP30, CP50, CP60, CP70, CP90), CUNO Incorporated HP series depth filters (e.g. HP10, HP30, HP50, HP60, HP70, HP90), CUNO Incorporated Calif. series depth filters (e.g. CA10, CA30, CA50, CA60, CA70, CA90), CUNO Incorporated SP series depth filters (e.g. SP10, SP30, SP50, SP60, SP70, SP90), CUNO Delipid and Delipid Plus filters, Millipore Corporation CE series depth filters (e.g. CE15, CE20, CE25, CE30, CE35, CE40, CE45, CE50, CE70, CE75), Millipore Corporation DE series depth filters (e.g. DE25, DE30, DE35, DE40, DE45, DE50, DE55, DE560, DE65, DE70, DE75), Millipore Corporation HC filters (e.g. A1HC, B1 HC, COHC), CUNO PolyNet™ Filters (e.g. PolyNet™ PB P050, P100, P200, P300, P400, P500, P700), Millipore Clarigard and Polygard filters, CUNO Life Assure filters, ManCel Associates depth filters (e.g. PR 12 UP, PR12, PR 5 UP); and PALL or SeitzSchenk Incorporated filters. In order to improve the clarification capacity of the available depth filtration units, it can be useful to couple two or more units with decreasing pore sizes. In this embodiment, the mixture to be clarified passes through the first depth filtration unit where the biggest contaminants are retained and subsequently passes through the second depth filtration unit. With this regard, according to a preferred embodiment of the invention, the clarification is performed by depth filtration, preferably over filters having a pore size of 8 μm coupled to filters having a pore size of 5 μm. Preferred filters having a pore size of 8 μm and 5 μm used according to the present invention are Sartopure® filters commercially available from Sartorius (Sartopure® PP2). According to the invention, the depth filtration is preferably performed at a flow rate of 1 L/minute.
A concentration which can be performed by e.g. microfiltration or ultrafiltration. Microfiltration is a pressure driven membrane process that concentrates and purifies large molecules. More specifically, a solution is passed through filters whose pore size has been chosen to reject the viruses in the retentate and allow small molecules (e.g. proteins) to pass through the filters into the permeate. Microfiltration reduces the volume of the extraction solution. With this regard, the microfiltration is therefore performed by using filters having a pore size lower than 0.2 μm, preferably a pore size comprised between 0.01 and 0.15 μm, more preferably between 0.09 and 0.15 μm, and even more preferably a pore size of 0.1 μm. Filters used according to the invention are preferably autoclavable commercially available filters such as for instance Prostak Microfiltration Modules (Millipore) wherein Prostak Microfiltration Module PSVVAG021, PSVVAG041 and SK2P12E1 are preferred.
A diafiltration which is an improvement of microfiltration (as previously described) and involves diluting said fraction comprising the viruses with a solution to effect a reduction in the concentration of the impurities in said fraction. The dilution of the fraction comprising the viruses allows washing out more of the impurities from said fraction. It is understood that the diafiltration may be carried out in a batch mode, semi-continuous mode, or a continuous mode. The diafiltration can be advantageously used to change the buffer in which the virus is comprised. For example, it can be useful to exchange the buffer used in the purification process against a pharmaceutically acceptable buffer. According to the invention, the microfiltration is performed by using filters having a pore size lower than 0.2 μm, preferably a pore size comprised between 0.01 and 0.15 μm, more preferably between 0.09 and 0.15 μm, and even more preferably a pore size of 0.1 μm. Filters used according to the invention are preferably autoclavable commercially available filters such as for instance Prostak Microfiltration Modules (Millipore) wherein Prostak Microfiltration Module PSVVAG021, PSVVAG041 and SK2P12E1 are preferred.
A chromatography using a cation or an anion exchange adsorbent, and preferably an anion exchange adsorbent. According to the invention, the functional groups of the anion exchange adsorbent can be primary, secondary, tertiary or quaternary amino group such as for instance dimethylaminoethyl (DMAE), diethylaminoethyl (DEAE), trimethylaminoethyl (TMAE), triethylaminoethyl (TEAE), the group —R—CH(OH)—CH₂—N+—(CH₃)₃ (also named Q group; see Streamline® resins, Pharmacia) or other groups such as for instance polyethyleneimine (PEI) that already have or will have a formal positive charge within the pH range of 7.0 to 9.0. Preferred functional groups of the anion exchange adsorbent are selected from the group consisting of dimethylaminoethyl (DMAE), diethylaminoethyl (DEAE), trimethylaminoethyl (TMAE) and triethylaminoethyl (TEAE), and are more preferably trimethylaminoethyl (TMAE). The anion exchange adsorbent can consist in, but is not limited to, e.g. a beads-formed matrix or a membrane.
   According to a preferred embodiment of the invention, the anion exchange adsorbent consists in a beads-formed matrix. Matrix can be e.g. agarose, hydrophilic polymer, cellulose, dextran or silica. Chains (e.g. dextran chains) are coupled to the matrix. Functional groups as previously described are attached to the chains through chemically stable bonds (e.g. ether bonds). Preferred functional groups of the beads-formed matrix are trimethylaminoethyl (TMAE). Anion exchange adsorbents consisting in beads-formed matrix used according to the invention are preferably autoclavable such as for instance UNOsphere® Q (BioRad), UNOsphere® S (BioRad), STREAMLINE™ Q Sepharose® XL (Amersham Biosciences), STREAMLINE™ SP Sepharose® XL (Amersham Biosciences) or BioSepra® Q hyperZ (Pall Corporation). Preferred autoclavable anion exchange adsorbent consisting in a beads-formed matrix according to the present invention is UNOsphere® Q (BioRad). UNOsphere® Q (BioRad) consists in hydrophilic spherical polymeric beads having a diameter of 120 μm and carrying trimethylaminoethyl (TMAE) functional groups.
   According to another preferred embodiment of the invention, the anion exchange adsorbent consists in a membrane. Functional groups of the membrane can be as previously described. Preferred functional groups of the membrane are trimethylaminoethyl (TMAE). According to a preferred embodiment of the invention, the membrane used has a pore size lower than the size of the virus. With this regard, when the virus is an poxvirus (which have a size of 200 nm), the membrane has a pore size comprised between 1 and 5 μm, and preferably a pore size of 3 μm. Anion exchange adsorbents consisting in membranes used according to the invention are preferably autoclavable such as for instance Sartobind® 75 Q (Sartorius).
A gel filtration: According to the invention, the sample containing the virus is treated on a solid support comprising beads having a diameter comprised between 3 and 160 μm, advantageously between 80 and 160 μm, preferably between 40 and 105 μm, more preferably between 25 and 75 μm, more preferably between 20 and 80 μm, and even more preferably between 20 and 60 μm. According to the invention, said support has a porosity closed to the diameter of the virus (e.g. 200-300 nm for poxvirus) so that the latter does not penetrate into the beads. On the other hand, the molecules which are smaller in size penetrate into the beads and the migration thereof is slowed. The supports used for gel filtration can be based e.g. on agarose, dextran, acrylamide, silica, ethylene glycol/methacrylate copolymers, or mixtures thereof such as for instance mixtures of agarose and dextran. According to the invention, the supports are preferably used without functional groups. Gel filtration chromatography supports are commercially available such as for instance:
   Ethylene glycol/methacrylate gel filtration chromatography supports (e.g. Toyopearl® HW 55, Toyopearl® HW 65 and Toyopearl® HW 75, having a bead diameter comprised between 20 and 60 μm, Tosohaas);
   Allyl dextran/methylene bisacrylamide gel filtration chromatography supports (e.g. Sephacryl™ S300 HR having a bead diameter comprised between 25 and 75 μm; Sephacryl™ S400 HR having a bead diameter comprised between 25 and 75 μm; Sephacryl™ S500 HR having a bead diameter comprised between 25 and 75 μm; Sephacryl™ S1000 SF having a bead diameter comprised between 40 and 105 μm, all from Pharmacia);
   N-acrylaminohydroxypropanediol gel filtration chromatography supports (e.g. Trisacryl having a bead diameter comprised between 80 and 160 μm, Biosepra);
   Agarose gel filtration chromatography supports (e.g. Macro-Prep SE having a bead diameter comprised between 20 and 80 μm, Bio-Rad).
Ethylene glycol/methacrylate gel filtration chromatography supports (e.g. Toyopearl® HW 55, Toyopearl® HW 65 and Toyopearl® HW 75, having a bead diameter comprised between 20 and 60 μm, Tosohaas) are preferred.

The method for producing a wild type, an attenuated and/or a recombinant virus comprising a step of preparation of cells from chicken embryos using an enzymatic composition of the invention, may also comprise a step of incubation in presence of one or more nucleases. Step of incubation in presence of one or more nucleases (i.e. endonuclease or exonucleases) is performed in order to degrade the nucleic acids (e.g. DNA; RNA) present in solution. Nucleases preferably used according to the present invention are endonucleases. Endonucleases can be classified based on their substrates as follows: deoxyribonucleases (DNases) which degrade DNA; ribonucleases (RNases) which degrade RNA; and endonucleases that degrade DNA and RNA. Endonucleases DNases include but are not limited to DNase I, DNase II and endodeoxyribonuclease IV. Endonucleases RNases include but are not limited to RNase I, RNase III, RNAse E, RNAse F and RNAse P. Endonucleases that degrade DNA and RNA include but are not limited to Benzonase®. In a preferred embodiment of the invention, step of incubating the viruses produced is performed in presence of Benzonase®. Benzonase® degrades nucleic acid (e.g. DNA; RNA) by hydrolyzing internal phosphodiester bonds between specific nucleotides. Upon complete digestion, all free nucleic acids (e.g. DNA; RNA) present in solution are reduced to 5′-monophosphate terminated oligonucleotides which are 3 to 8 bases in length. Benzonaze® has no proteolytic activity. Benzonaze® used according to the present invention is preferably pharmaceutically acceptable. Pharmaceutically acceptable Benzonaze® are commercially available (e.g. Eurogentec under the reference ME-0280-10; Merck under the reference e.g. 1.01653.0001). According to the invention, the concentration of nuclease(s) used is in a range of 5 to 100 U/ml, preferably in a range of 5 to 50 U/ml, and more preferably 10 U/ml.

The method for producing a wild type, an attenuated and/or a recombinant virus according to the invention are suitable for an aseptic industrial-scale manufacturing process to ensure a full compliance with regulatory requirements regarding sterility of vaccines.

As used throughout in the entire application, “virus” includes but is not limited to poxvirus, adenovirus, adenovirus-associated virus, retrovirus, herpesvirus, alphavirus, foamy virus, flu virus (such as e.g. influenza virus), flavivirus (such as e.g. yellow fever virus, japanese encephalitis virus, dengue virus, tick-borne encephalitis virus or west nile virus), measles virus, rubella virus, alphavirus (such as e.g. ross river virus, chikungunya virus), hepatitis virus, rhinovirus, reovirus (such as e.g. colorado tick fever) or foamy virus.

In a preferred embodiment of the invention, the virus is a poxvirus. Poxviruses are complex enveloped viruses having a diameter comprised between 200 and 300 nm that distinguish them principally by their unusual morphology, their large DNA genome and their cytoplasmic site of replication. According to the invention the poxviruses can be indifferently immature viruses (IV), intracellular mature viruses (IMV), intracellular enveloped viruses (IEV), cell-associated enveloped viruses (CEV) or extracellular enveloped viruses (EEV) (SMITH et al. (2002), J. Gen. Virol., 83, 2915-2931). Poxviruses as used in the present invention refer preferably to poxviruses of the subfamily Chordopoxviruses (vertebrate poxviruses) (Fields Virology/eds.: FIELDS, B. N., KNIPE, D. M., HOWLEY, P. M.; 3rd ed/ISBN 0-7817-0253-4; Chapter 83). Chordopoxviruses include but are not limited to poxviruses of the genus of Orthopoxviruses, Parapoxviruses, Avipoxviruses, Capripoxviruses, Lepripoxviruses, Suipoxviruses, Molluscipoxviruses or Yatapoxviruses. Preferred Chordopoxviruses according to the invention are Orthopoxviruses. Orthopoxviruses include but are limited to variola viruses, Vaccinia Viruses (VV) such as for instance the Vaccinia virus strains Elstree, Western Reserve, Wyeth, NYVAC, NYCBOH, Paris, Copenhagen (GOEBLE et al. (1990); Genbank accession number M35027.1), or their derivatives such as for instance a modified Vaccinia Virus Ankara (MVA) in particular MVA 575 (ECACC V00120707) and MVA-BN (ECACC V00083008). The genome of several members of poxviruses, including the Copenhagen Vaccinia Virus (VV) strain (GOEBEL et al., 1990, Virol. 179, 247-266 and 517-563; JOHNSON et al., 1993, Virol. 196, 381-401) and the modified Vaccinia Virus Ankara (MVA) strain (ANTOINE et al., 1998, Virol. 244, 365-396), have been mapped and sequenced. VV has a double-stranded DNA genome of about 192 kb coding for about 200 proteins of which approximately 100 are involved in virus assembly. MVA is a highly attenuated Vaccinia Virus strain generated by more than 500 serial passages of the Ankara strain of Vaccinia Virus on chicken embryo fibroblasts (MAYR et al., 1975, Infection 3, 6-16). The MVA virus was deposited before Collection Nationale de Cultures de Microorganismes (CNCM) under depositary N⁶⁰² I-721. Determination of the complete sequence of the MVA genome and comparison with the Copenhagen VV genome allows the precise identification of the alterations which occurred in the viral genome and the definition of seven deletions (I to VII) and numerous mutations leading to fragmented ORFs (Open Reading Frame) (ANTOINE et al., 1998, Virology 244, 365-396).

In a preferred embodiment of the invention, the virus is a poxvirus and preferably an Orthopoxvirus.

In a particular embodiment of the invention, the Orthopoxvirus is a Vaccinia Virus (VV). Preferred VV according to the invention are VV as described for instance in patent applications PCT/EP2008/009720 or PCT/EP2008/009721 describing respectively VV comprising defective 14L and/or F4L gene(s) and VV comprising a defective F2L gene.

In another particular embodiment of the invention, the Orthopoxvirus is a modified Vaccinia Virus Ankara (MVA). Preferred MVA according to the present invention are MVA as deposited before Collection Nationale de Cultures de Microorganismes (CNCM) under depositary N⁶⁰² I-721, MVA 575 (ECACC V00120707) and MVA-BN (ECACC V00083008).

In a preferred embodiment, the invention relates to a method for producing a wild type, an attenuated and/or a recombinant poxvirus comprising a step of preparation of cells from embryo using an enzymatic composition as previously described and further steps as described in WO 07/147,528 patent application incorporated herein by reference. With this regard, the invention therefore relates to a method for producing a wild type, an attenuated and/or a recombinant poxvirus comprising the steps of:
a) preparing cells from embryos using an enzymatic composition as previously described;
b) infecting said cell culture (as described in WO 07/147,528 patent application incorporated herein by reference);
c) culturing said infected cells for an appropriate period of time (as described in WO 07/147,528 patent application incorporated herein by reference);
d) recovering the poxyiral particles produced from the culture supernatant and/or the packaging cells (as described in WO 07/147,528 patent application incorporated herein by reference);
e) clarifying the mixture obtained by depth filtration, preferably over filters having a pore size of 8 μm coupled to filters having a pore size of 5 μm (as described in WO 07/147,528 patent application incorporated herein by reference);
f) concentrating the mixture obtained by microfiltration, preferably over filters having a pore size comprised between 0.01 and 0.15 μm, and preferably over filters having a pore size of 0.1 μm (as described in WO 07/147,528 patent application incorporated herein by reference); and
g) diafiltring the mixture obtained, preferably over filters having a pore size comprised between 0.01 and 0.15 μm, and preferably over filters having a pore size of 0.1 μm (as described in WO 07/147,528 patent application incorporated herein by reference).

In another preferred embodiment, the invention relates to a method for producing a wild type, an attenuated and/or a recombinant Orthopoxvirus comprising a step of preparation of cells from embryos using an enzymatic composition as previously described and further steps as described in Method A of EP09305422.9 patent application incorporated herein by reference. With this regard, the invention therefore relates to a method for producing a wild type, an attenuated and/or a recombinant Orthopoxvirus comprising the steps of:
a) preparing cells from embryos using an enzymatic composition as previously described;
b) infecting the cells with an Orthopoxvirus (as described in Method A of EP09305422.9 patent application incorporated herein by reference);
c) culturing the infected cells until progeny Orthopoxvirus is produced (as described in Method A of EP09305422.9 patent application incorporated herein by reference);
d) incubation in presence of one or more nucleases (as described in Method A of EP09305422.9 patent application incorporated herein by reference);
e) recovering the Orthopoxviruses from the culture supernatant and/or the cells (as described in Method A of EP09305422.9 patent application incorporated herein by reference);
f) adding monovalent salts (e.g. NaCl; KCl) to the Orthopoxviruses recovered in step e) under suitable conditions to inhibit the nuclease(s) activity and to avoid the adsorption of said Orthopoxviruses to the anion exchange adsorbent in step g) (as described in Method A of EP09305422.9 patent application incorporated herein by reference);
g) contacting the mixture obtained in step f) with an anion exchange adsorbent (e.g. a beads-formed matrix or a membrane having functional groups being primary, secondary, tertiary or quaternary amino group such as for instance dimethylaminoethyl (DMAE), diethylaminoethyl (DEAE), trimethylaminoethyl (TMAE), triethylaminoethyl (TEAE), the group —R—CH(OH)—CH₂—N+—(CH₃)₃ (also named Q group; see Streamline® resins, Pharmacia) or other groups such as for instance polyethyleneimine (PEI) that already have or will have a formal positive charge within the pH range of 7.0 to 9.0) under suitable conditions to allow the capture of nucleic acids (as described in Method A of EP09305422.9 patent application incorporated herein by reference);
h) clarifying the mixture obtained in step g) under suitable conditions to allow the withdrawal of the cellular debris (as described in Method A of EP09305422.9 patent application incorporated herein by reference);
i) washing of the anion exchange adsorbent with a solution comprising monovalent salts (e.g. NaCl; KCl) under suitable conditions to recover the remained Orthopoxviruses in the flow through (as described in Method A of EP09305422.9 patent application incorporated herein by reference);
j) concentrating the flow through obtained in step h) and the flow through obtained in step i) (as described in Method A of EP09305422.9 patent application incorporated herein by reference);
k) diafiltrating the fraction comprising the Orthopoxviruses obtained in step j) (as described in Method A of EP09305422.9 patent application incorporated herein by reference); and optionally
l) a step of gel filtration followed by a step of diafiltration (as described in Method A of EP09305422.9 patent application incorporated herein by reference).

In another preferred embodiment, the invention relates to a method for producing a wild type, an attenuated and/or a recombinant Orthopoxvirus comprising a step of preparation of cells from embryos using an enzymatic composition as previously described and further steps as described in Method B of EP09305422.9 patent application incorporated herein by reference. With this regard, the invention therefore relates to a method for producing a wild type, an attenuated and/or a recombinant Orthopoxvirus comprising the steps of:
a′) preparing cells from embryos using an enzymatic composition as previously described;
b′) infecting the cell culture with an Orthopoxvirus (as described in Method B of EP09305422.9 patent application incorporated herein by reference);
c′) culturing the infected cells until progeny Orthopoxvirus is produced (as described in Method B of EP09305422.9 patent application incorporated herein by reference);
d′) incubation in presence of one or more nucleases (as described in Method B of EP09305422.9 patent application incorporated herein by reference);
e′) recovering the Orthopoxviruses from the culture supernatant and/or the packaging cells (as described in Method B of EP09305422.9 patent application incorporated herein by reference);
f′) incubating the Orthopoxviruses recovered in step e′) in presence of:
   1. one or more agents capable to inhibit the nuclease(s) activity (e.g. chelating agents such as e.g. ethylenediamine tetraacetate (EDTA); monovalent salts such as e.g. NaCl or KCl), and optionally
   2. one or more stabilizers (e.g. saccharides such as e.g. sucrose or trehalose; amino acids; detergents such as Tween; salts such as e.g. NaCl or KCl)
(as described in Method B of EP09305422.9 patent application incorporated herein by reference);
g′) contacting the mixture obtained in step f′) with an anion exchange adsorbent (e.g. a beads-formed matrix or a membrane having functional groups being primary, secondary, tertiary or quaternary amino group such as for instance dimethylaminoethyl (DMAE), diethylaminoethyl (DEAE), trimethylaminoethyl (TMAE), triethylaminoethyl (TEAE), the group —R—CH(OH)—CH₂—N+—(CH₃)₃ (also named Q group; see Streamline® resins, Pharmacia) or other groups such as for instance polyethyleneimine (PEI) that already have or will have a formal positive charge within the pH range of 7.0 to 9.0) under suitable conditions to allow the capture of said Orthopoxviruses and nucleic acids (as described in Method B of EP09305422.9 patent application incorporated herein by reference);
h′) clarifying the mixture obtained in step g′) under suitable conditions to allow the withdrawal of the cellular debris (as described in Method B of EP09305422.9 patent application incorporated herein by reference);
i′) eluting the Orthopoxviruses with a solution comprising monovalent salts;
j′) concentrating the mixture obtained in step i′) (as described in Method B of EP09305422.9 patent application incorporated herein by reference);
k′) diafiltrating the fraction comprising the Orthopoxviruses obtained in step j′); and optionally
l′) a step of gel filtration followed by a step of diafiltration (as described in Method B of EP09305422.9 patent application incorporated herein by reference).

In another preferred embodiment, the invention relates to a method for producing a wild type, an attenuated and/or a recombinant Orthopoxvirus comprising a step of preparation of cells from embryos using an enzymatic composition as previously described and further steps as described in Method C of PCT/EP2010/056491 patent application incorporated herein by reference. With this regard, the invention therefore relates to a method for producing a wild type, an attenuated and/or a recombinant Orthopoxvirus comprising the steps of:
a″) preparing cells from embryos using an enzymatic composition as previously described;
b″) infecting the packaging cell culture with an Orthopoxvirus (as described in Method C of PCT/EP2010/056491 patent application incorporated herein by reference);
c″) culturing the infected packaging cells until progeny Orthopoxvirus is produced (as described in Method C of PCT/EP2010/056491 patent application incorporated herein by reference);
d″) incubation in presence of one or more nucleases (as described in Method C of PCT/EP2010/056491 patent application incorporated herein by reference);
e″) recovering the Orthopoxviruses from the culture supernatant and/or the packaging cells (as described in Method C of PCT/EP2010/056491 patent application incorporated herein by reference);
f″) incubating the Orthopoxviruses recovered in step e″) in presence of:
   1. one or more agents capable to inhibit the nuclease(s) activity, and optionally
   2. one or more stabilizers;
(as described in Method C of PCT/EP2010/056491 patent application incorporated herein by reference);
g″) contacting the mixture obtained in step f″) with an anion exchange adsorbent (e.g. a beads-formed matrix or a membrane having functional groups being primary, secondary, tertiary or quaternary amino group such as for instance dimethylaminoethyl (DMAE), diethylaminoethyl (DEAE), trimethylaminoethyl (TMAE), triethylaminoethyl (TEAE), the group —R—CH(OH)—CH₂—N+—(CH₃)₃ (also named Q group; see Streamline® resins, Pharmacia) or other groups such as for instance polyethyleneimine (PEI) that already have or will have a formal positive charge within the pH range of 7.0 to 9.0) under suitable conditions to allow the capture of said Orthopoxviruses and nucleic acids (as described in Method C of PCT/EP2010/056491 patent application incorporated herein by reference);
h″) clarifying the mixture obtained in step g″) under suitable conditions to allow the withdrawal of the cellular debris (as described in Method C of PCT/EP2010/056491 patent application incorporated herein by reference);
i″) eluting the Orthopoxviruses with a solution comprising monovalent salts (as described in Method C of PCT/EP2010/056491 patent application incorporated herein by reference);
j″) adding monovalent salts to the Orthopoxviruses eluted in step i″) in order to avoid the adsorption of said Orthopoxviruses to the anion exchange adsorbent in step k″) (as described in Method C of PCT/EP2010/056491 patent application incorporated herein by reference);
k″) contacting the mixture obtained in step j″) with an anion exchange adsorbent (e.g. a beads-formed matrix or a membrane having functional groups being primary, secondary, tertiary or quaternary amino group such as for instance dimethylaminoethyl (DMAE), diethylaminoethyl (DEAE), trimethylaminoethyl (TMAE), triethylaminoethyl (TEAE), the group —R—CH(OH)—CH₂—N+—(CH₃)₃ (also named Q group; see Streamline® resins, Pharmacia) or other groups such as for instance polyethyleneimine (PEI) that already have or will have a formal positive charge within the pH range of 7.0 to 9.0) under suitable conditions to allow the capture of nucleic acids (as described in Method C of PCT/EP2010/056491 patent application incorporated herein by reference);
l″) washing of the anion exchange adsorbent with a solution comprising monovalent salts under suitable conditions to recover the remained Orthopoxviruses in the flow through (as described in Method C of PCT/EP2010/056491 patent application incorporated herein by reference);
m″) concentrating the flow through obtained in step l″) (as described in Method C of PCT/EP2010/056491 patent application incorporated herein by reference);
n″) diafiltrating the fraction comprising the Orthopoxviruses obtained in step m″) (as described in Method C of PCT/EP2010/056491 patent application incorporated herein by reference), and optionally
o″) a step of gel filtration followed by a step of diafiltration (as described in Method C of PCT/EP2010/056491 patent application incorporated herein by reference).

As used throughout the entire application, “attenuated virus” refers to any virus that haS been modified so that its pathogenicity in the intended subject is substantially reduced. Preferably, the virus is attenuated to the point it is nonpathogenic from a clinical standpoint, i.e. that subjects exposed to the virus do not exhibit a statistically significant increased level of pathology relative to control subjects.

As used throughout the entire application, “recombinant “virus” refers to a virus comprising an exogenous sequence inserted in its genome. As used herein, an exogenous sequence refers to a nucleic acid which is not naturally present in the parent virus.

In one embodiment, the exogenous sequence encodes a molecule having a directly or indirectly cytotoxic function. By “directly or indirectly” cytotoxic, we mean that the molecule encoded by the exogenous sequence may itself be toxic (for example ricin, tumour necrosis factor (TNF), interleukin-2 (IL2), interferon-gamma (IFNγ), ribonuclease, deoxyribonuclease, Pseudomonas exotoxin A) or it may be metabolised to form a toxic product, or it may act on something else to form a toxic product. The sequence of ricin cDNA is disclosed in Lamb et al (Eur. J. Biochem., 1985, 148, 265-270).

In a preferred embodiment of the invention, the exogenous sequence is a suicide gene. A suicide gene encodes a protein able to convert a relatively non-toxic prodrug to a toxic drug. For example, the enzyme cytosine deaminase converts 5-fluorocytosine (5-FC) to 5-fluorouracil (5-FU) (Mullen et al (1922) PNAS 89, 33); the herpes simplex enzyme thymidine kinase sensitises cells to treatment with the antiviral agent ganciclovir (GCV) or aciclovir (Moolten (1986) Cancer Res. 46, 5276; Ezzedine et al (1991) New Biol 3, 608). The cytosine deaminase of any organism, for example E. coli or Saccharomyces cerevisiae, may be used. Thus, in preferred embodiment of the invention, the suicide gene encodes a protein having a cytosine deaminase activity, and more preferably FCU1 protein or FCU1-8 protein covered by patent applications WO 99/54481, WO 05/07857, PCT/EP2008/009720 and PCT/EP2008/009721 incorporated herein by reference.

With this regard, preferred recombinant viruses produced according to the method of the invention are:
MVA-FCU1 (see WO 99/54481) also called TG4023;
MVA-FCU1-8 (see WO 05/07857); and
VV-FCU1 wherein said VV comprises more particularly a defective I4L and/or F4L gene, and a defective J2R gene (see PCT/EP2008/009720 and PCT/EP2008/009721).

Other examples of pro-drug/enzyme combinations include those disclosed by Bagshawe et al (WO 88/07378), namely various alkylating agents and the Pseudomonas spp. CPG2 enzyme, and those disclosed by Epenetos & Rowlinson-Busza (WO 91/11201), namely cyanogenic pro-drugs (for example amygdalin) and plant-derived beta-glucosidases. Enzymes that are useful in this embodiment of the invention include, but are not limited to, alkaline phosphatase useful for converting phosphate-containing prodrugs into free drugs; arylsulfatase useful for converting sulfate-containing prodrugs into free drugs; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as beta-galactosidase and neuraminidase useful for converting glycosylated prodrugs into free drugs; beta-lactamase useful for converting drugs derivatized with beta-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as abzymes, can be used to convert the prodrugs of the invention into free active drugs (Massey R. et al., Nature, 1987, 328, 457-458). Similarly, prodrugs include, but are not limited to, the above-listed prodrugs, e.g., phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted. Examples of cytotoxic drugs that can be derivatized into a prodrug form for use in this invention include, but are not limited to etoposide, teniposide, adriamycin, daunomycin, caminomycin, aminopterin, dactinomycin, mitomycins, cis-platinum and cis-platinum analogues, bleomycins, esperamicins (see for example U.S. Pat. No. 4,675,187), 5-fluorouracil, melphalan and other related nitrogen mustards.

In a further embodiment the exogenous gene encodes a ribozyme capable of cleaving targeted RNA or DNA. The targeted RNA or DNA to be cleaved may be RNA or DNA which is essential to the function of the cell and cleavage thereof results in cell death or the RNA or DNA to be cleaved may be RNA or DNA which encodes an undesirable protein, for example an oncogene product, and cleavage of this RNA or DNA may prevent the cell from becoming cancerous.

In a still further embodiment the exogenous gene encodes an antisense RNA. By “antisense RNA” we mean an RNA molecule which hybridises to, and interferes with the expression from an mRNA molecule encoding a protein or to another RNA molecule within the cell such as pre-mRNA or tRNA or rRNA, or hybridises to, and interferes with the expression from a gene.

In another embodiment of the invention, the exogenous sequence replaces the function of a defective gene in the target cell. There are several thousand inherited genetic diseases of mammals, including humans, which are caused by defective genes. Examples of such genetic diseases include cystic fibrosis, where there is known to be a mutation in the CFTR gene; Duchenne muscular dystrophy, where there is known to be a mutation in the dystrophin gene; sickle cell disease, where there is known to be a mutation in the HbA gene. Many types of cancer are caused by defective genes, especially protooncogenes, and tumour-suppressor genes that have undergone mutation. Examples of protooncogenes are ras, src, bcl and so on; examples of tumour-suppressor genes are p53 and Rb.

In a further embodiment of the invention, the exogenous sequence encodes a Tumor Associated Antigen (TAA). TAA refers to a molecule that is detected at a higher frequency or density in tumor cells than in non-tumor cells of the same tissue type. Examples of TAA includes but are not limited to CEA, MART1, MAGE1, MAGE3, GP-100, MUC1 (see WO 92/07000, WO 95/09241 and Rochlitz et al. J Gene Med. 2003 August; 5(8):690-9 incorporated herein by reference), MUC2, pointed mutated ras oncogene, normal or point mutated p53, overexpressed p53, CA-125, PSA, C-erb/B2, BRCA I, BRCA II, PSMA, tyrosinase, TRP1, TRP2, NY-ESO-1, TAG72, KSA, HER-2/neu, bcr-abl, pax3-fkhr, ews-fli-1, surviving and LRP. According to a more preferred embodiment the TAA is MUC1.

In another embodiment of the invention, the exogenous gene encodes an antigen. As used herein, “antigen” refers to a ligand that can be bound by an antibody; an antigen need not itself be immunogenic. Preferably the antigen is derived from a virus such as for example HIV-1, (such as gp120 or gp 160), any of Feline Immunodeficiency virus, human or animal herpes viruses, such as gD or derivatives thereof or Immediate Early protein such as ICP27 from HSV1 or HSV2, cytomegalovirus (such as gB or derivatives thereof), Varicella Zoster Virus (such as gpl, II or III), or from a hepatitis virus such as hepatitis B virus (HBV) for example Hepatitis B Surface antigen or a derivative thereof, hepatitis A virus (HAV), hepatitis C virus (HCV; see WO 04/111082; preferentially non structural HCV protein from genotype 1 b strain ja), and hepatitis E virus (HEV), or from other viral pathogens, such as Respiratory Syncytial Virus, Human Papilloma Virus (HPV; see WO 90/10459, WO 95/09241, WO 98/04705, WO 99/03885 WO 07/121,894 and WO 07/121,894; E6 and E7 protein from the HPV16 strain are preferred; see also Liu et al. Proc Natl Acad Sci USA. 2004 Oct. 5; 101 Suppl 2:14567-71) or Influenza virus, or derived from bacterial pathogens such as Salmonella, Neisseria, Borrelia (for example OspA or OspB or derivatives thereof), or Chlamydia, or Bordetella for example P.69, PT and FHA, or derived from parasites such as plasmodium or Toxoplasma. According to a more preferred embodiment the antigen is selected from HCV or HPV.

With this regard, preferred recombinant virus produced according to the method of the invention is MVA-HCV (see WO 04/111082) also called TG4040.

The recombinant virus can comprise more than one exogenous sequence and each exogenous sequence can encodes more than one molecule. For example, it can be useful to associate in a same recombinant virus, an exogenous sequenced encoding e.g. a TAA (as previously described) or an antigen (as previously described) with an exogenous sequence encoding a cytokine (e.g. interleukin (IL as for instance IL2); tumour necrosis factor (TNF); interferon-(IFN); colony stimulating factor (CSF)).

With this regard, preferred recombinant viruses produced according to the method of the invention are:
MVA-[MUC1-IL2] (see WO 92/07000 and WO 95/09241) also called TG4010; and
MVA-[HPV-IL2] (see WO 90/10459, WO 95/09241, WO 98/04705, WO 99/03885, WO 07/121,894 and WO 07/121,894) also called TG4001.

Advantageously, the recombinant virus further comprises the elements necessary for the expression of the exogenous sequence(s). The elements necessary for the expression comprise of the set of elements allowing the transcription of a nucleotide sequence to RNA and the translation of a mRNA to a polypeptide, in particular the promoter sequences and/or regulatory sequences which are effective in the cell to be infected by the recombinant virus of the invention, and optionally the sequences required to allow the excretion or the expression at the surface of the cells for said polypeptide. These elements may be inducible or constitutive. Of course, the promoter is adapted to the recombinant virus selected and to the host cell. There may be mentioned, by way of example, the Vaccinia Virus promoters p7.5K pH5R, pK1L, p28, p11 or a combination of said promoters. The literature provides a large amount of information relating to such promoter sequences. The elements necessary can, in addition, include additional elements which improve the expression of the exogenous sequence or its maintenance in the host cell. There may be mentioned in particular the intron sequences (WO 94/29471), secretion signal sequences, nuclear localization sequences, internal sites for reinitiation of translation of the IRES type, poly A sequences for termination of transcription.

The present invention also relates to a purified wild type, attenuated and/or recombinant virus obtained by the method as previously described for use as a pharmaceutical composition, preferably as a vaccine.

As used herein, a “pharmaceutical composition” refers to a composition comprising a pharmaceutically acceptable carrier. Said pharmaceutically acceptable carrier is preferably isotonic, hypotonic or weakly hypertonic and has a relatively low ionic strength, such as for example a sucrose solution. Moreover, such a carrier may contain any solvent, or aqueous or partially aqueous liquid such as nonpyrogenic sterile water. The pH of the pharmaceutical composition is, in addition, adjusted and buffered so as to meet the requirements of use in vivo. The pharmaceutical compositions may also include a pharmaceutically acceptable diluent, adjuvant or excipient, as well as solubilizing, stabilizing and preserving agents. For injectable administration, a formulation in aqueous, nonaqueous or isotonic solution is preferred. It may be provided in a single dose or in a multidose in liquid or dry (powder, lyophilisate and the like) form which can be reconstituted at the time of use with an appropriate diluent.

The present invention also relates to a purified wild type, attenuated and/or recombinant virus obtained by the method as previously described for the treatment and/or the prevention a cancer, an infectious disease and/or an autoimmune disorder.

As used herein, “cancer” refers but is not limited to lung cancer (e.g. small cell lung carcinomas and non-small cell lung), bronchial cancer, oesophageal cancer, pharyngeal cancer, head and neck cancer (e.g. laryngeal cancer, lip cancer, nasal cavity and paranasal sinus cancer and throat cancer), oral cavity cancer (e.g. tongue cancer), gastric cancer (e.g. stomach cancer), intestinal cancer, gastrointestinal cancer, colon cancer, rectal cancer, colorectal cancer, anal cancer, liver cancer, pancreatic cancer, urinary tract cancer, bladder cancer, thyroid cancer, kidney cancer, carcinoma, adenocarcinoma, skin cancer (e.g. melanoma), eye cancer (e.g. retinoblastoma), brain cancer (e.g. glioma, medulloblastoma and cerebral astrocytoma), central nervous system cancer, lymphoma (e.g. cutaneous B-cell lymphoma, Burkitt's lymphoma, Hodgkin's syndrome and non-Hodgkin's lymphoma), bone cancer, leukaemia, breast cancer, genital tract cancer, cervical cancer (e.g. cervical intraepithelial neoplasia), uterine cancer (e.g. endometrial cancer), ovarian cancer, vaginal cancer, vulvar cancer, prostate cancer, testicular cancer. “Cancers” also refer to virus-induced tumors, including, but is not limited to papilloma virus-induced carcinoma, herpes virus-induced tumors, EBV-induced B-cell lymphoma, hepatitis B-induced tumors, HTLV-1-induced lymphoma and HTLV-2-induced lymphoma.

As used herein, “infectious disease” refers to any disease that is caused by an infectious organism. Infectious organisms include, but are not limited to, viruses (e.g. single stranded RNA viruses, single stranded DNA viruses, human immunodeficiency virus (HIV), hepatitis A, B, and C virus, herpes simplex virus (HSV), cytomegalovirus (CMV), respiratory syncytial virus (RSV), Epstein-Barr virus (EBV) or human papilloma virus (HPV)), parasites (e.g. protozoan and metazoan pathogens such as Plasmodia species, Leishmania species, Schistosoma species or Trypanosoma species), bacteria (e.g. Mycobacteria in particular, M. tuberculosis, Salmonella, Streptococci, E. coli or Staphylococci), fungi (e.g. Candida species or Aspergillus species), Pneumocystis carinii, and prions.

As used herein, “autoimmune disorder” refers to two general types: ‘Systemic autoimmune diseases’ (i.e., disorders that damage many organs or tissues), and ‘localized autoimmune diseases’ (i.e., disorders that damage only a single organ or tissue). However, the effect of ‘localized autoimmune diseases’, can be systemic by indirectly affecting other body organs and systems. ‘Systemic autoimmune diseases’ include but are not limited to rheumatoid arthritis which can affect joints, and possibly lung and skin; lupus, including systemic lupus erythematosus (SLE), which can affect skin, joints, kidneys, heart, brain, red blood cells, as well as other tissues and organs; scleroderma, which can affect skin, intestine, and lungs; Sjogren's syndrome, which can affect salivary glands, tear glands, and joints; Goodpasture's syndrome, which can affect lungs and kidneys; Wegener's granulomatosis, which can affect sinuses, lungs, and kidneys; polymyalgia rheumatica, which can affect large muscle groups, and temporal arteritis/giant cell arteritis, which can affect arteries of the head and neck. ‘Localized autoimmune diseases’ include but are not limited to Type 1 Diabetes Mellitus, which affects pancreas islets; Hashimoto's thyroiditis and Graves' disease, which affect the thyroid; celiac disease, Crohn's diseases, and ulcerative colitis, which affect the gastrointestinal tract; multiple sclerosis (MS) and Guillain-Barre syndrome, which affect the central nervous system; Addison's disease, which affects the adrenal glands; primary biliary sclerosis, sclerosing cholangitis, and autoimmune hepatitis, which affect the liver; and Raynaud's phenomenon, which can affect the fingers, toes, nose, ears.

The present invention also relates to a pharmaceutical composition, preferably a vaccine, comprising a purified wild type, attenuated and/or recombinant virus obtained by the method as previously described. According to the invention, said pharmaceutical composition is intended for the treatment and/or the prevention a cancer, an infectious disease and/or an autoimmune disorder.

The present invention also relates to the use of a purified wild type, attenuated and/or recombinant virus obtained by the method as previously described for the preparation of a pharmaceutical composition, preferably a vaccine, for the treatment and/or the prevention a cancer, an infectious disease and/or an autoimmune disorder.

The pharmaceutical composition and in particular the vaccine may be manufactured conventionally for administration by the local, parenteral or digestive route. The routes of administration may be for instance the intragastric, subcutaneous, intracardiac, intramuscular, intravenous, intraperitoneal, intratumor, intranasal, intrapulmonary or intratracheal route. For the latter three embodiments, administration by aerosol or instillation is advantageous. The administration may be made as a single dose or repeated once or several times after a certain time interval. The appropriate route of administration and dosage vary as a function of various parameters, for example, of the individual, of the disease to be treated or of the gene(s) of interest to be transferred. According to a first possibility, the pharmaceutical composition and in particular the vaccine may be administered directly in vivo (for example by intravenous injection, into an accessible tumor or at its periphery, subcutaneously for a therapeutic or prophylactic vaccination). It is also possible to adopt the ex vivo approach which consists in collecting cells from the patient (bone marrow stem cells, peripheral blood lymphocytes, muscle cells and the like), transfecting or infecting them in vitro according to prior art techniques and readministering them to the patient. It is moreover possible to envisage, where appropriate and without departing from the scope of the present invention, carrying out simultaneous or successive administrations, by different routes, of the various components contained in the pharmaceutical composition and in particular in the vaccine.

To illustrate the invention, the following examples are provided. The examples are not intended to limit the scope of the invention in any way.

### EXAMPLES

### Preparation of CEFs

Sixty six SPF eggs are incubated in for 1 minute in a 2% formol solution. After being rinsed with 70% ethanol, the eggs are opened, the embryos are extracted and the heads and feet are cut.

The embryos (not dissected) are then digested at 37° C. for 2 hours by one of enzymatic compositions as described in Table 1 (see below). The mixture obtained is filtrated using a seave made of inox (Fischer Bioblock, Cat No. A37532) to remove undigested tissues and the CEFs are collected by centrifugation (2300 rpm, 15 minutes).

The results obtained are depicted in Table 1 below:

| | CELLS EXTRACTED |
|---|---|
| ENZYMATIC COMPOSITIONS | PER EMBRYO |
| TrypLE ™ Select 30 mL/Embryo | 770 10⁶ |
| Dispase 10 mL/Embryo | |
| Collagenase type III 10 mL/Embryo | |
| TrypLE ™ Select 10 mL/Embryo | 710 10⁶ |
| Dispase 10 mL/Embryo | |
| Collagenase type III 10 mL/Embryo | |
| TrypLE ™ Select 15 mL/Embryo | 709 10⁶ |
| Dispase 10 mL/Embryo | |
| Accutase 10 mL/Embryo | |
| TrypLE ™ Select 15 mL/Embryo | 696 10⁶ |
| Dispase 10 mL/Embryo | |
| Collagenase type III 10 mL/Embryo | |
| Accutase 10 mL/Embryo | |
| TrypLE ™ Select 30 mL/Embryo | 605 10⁶ |
| Dispase 10 mL/Embryo | |
| Accutase 10 mL/Embryo | |
| TrypLE ™ Select 10 mL/Embryo | 602 10⁶ |
| Dispase 10 mL/Embryo | |
| Collagenase type III 5 mL/Embryo | |
| TrypLE ™ Select 30 mL/Embryo | 588 10⁶ |
| Dispase 10 mL/Embryo | |
| Collagenase type III 10 mL/Embryo | |
| Accutase 10 mL/Embryo | |
| TrypLE ™ Select 10 mL/Embryo | 583 10⁶ |
| Dispase 10 mL/Embryo | |
| Accutase 10 mL/Embryo | |
| TrypLE ™ Select 15 mL/Embryo | 567 10⁶ |
| Dispase 5 mL/Embryo | |
| TrypLE ™ Select 30 mL/Embryo | 560 10⁶ |
| Dispase 10 mL/Embryo | |
| TrypLE ™ Select 10 mL/Embryo | 546 10⁶ |
| Dispase 10 mL/Embryo | |
| Dispase 10 mL/Embryo | 542 10⁶ |
| Accutase 10 mL/Embryo | |
| TrypLE ™ Select 5 mL/Embryo | 531 10⁶ |
| Dispase 15 mL/Embryo | |
| TrypLE ™ Select 30 mL/Embryo | 291 10⁶ |
| Dispase 5 mL/Embryo | |
| Collagenase type III 5 mL/Embryo | |
| TrypLE ™ Select 30 mL/Embryo | 375 10⁶ |
| Accutase 15 mL/Embryo | |
| TrypLE ™ Select 30 mL/Embryo | 350 10⁶ |
| Collagenase type III 10 mL/Embryo | |
| TrypLE ™ Select 30 mL/Embryo | 345 10⁶ |

TrypLE ™ Select (Invitrogen, Cat. No. 12563-029);
Dispase (Invitrogen, Cat. No. 17105-041; 10 mg/mL in PBS);
Collagenase III (Gibco Invitrogen, Cat No. 17102; 20 mg/mL in PBS);
Accutase (Sigma, Cat. No. A-6964).

### Production of Viruses Using the Prepared CEFs.

CEFs (prepared as previously described with an enzymatic composition consisting in TrypLE™ Select (Invitrogen, Cat. No. 12563-029) 15 mL/Embryo and Dispase (Invitrogen, Cat. No. 17105-041; 10 mg/mL in PBS) 5 mL/Embryo) are then cultivated during 2 days in a serum free cell culture medium in humid atmosphere at 37° C. The serum free cell culture medium is then discarded and the CEFs are infected at MOI 0.05 with MVA virus (Collection Nationale de Cultures de Microorganismes (CNCM) under depositary N⁶⁰² I-721) expressing MUCI-IL-2. The infected CEFs are then incubated for 3 days at 36.5° C. The infectious titers were determined by plaque-assay on BHK21 cells. Briefly, dilutions of MVA samples were inoculated on monolayers of BHK21 cells prepared in 6-well plates. After 24 hours, the viral plaques were stained with immunoperoxidase reaction with rabbit antibodies raised against Vaccinia virus and counted. The titration were performed in triplicate, in three independent series. The viral titer obtained was: 6.3 10⁷ Plaque-Forming Unit (PFU)/mL.

All documents (e.g. patents, patent applications, publications) cited in the above specification are herein incorporated by reference. Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1.-21. (canceled)

22. A method for the digestion of chicken embryos to obtain a chicken cell preparation comprising using an enzymatic composition, wherein said enzymatic composition is selected from the group consisting of:
trypsin, dispase, and collagenase;
trypsin, dispase, and accutase;
trypsin, dispase, collagenase, and accutase;
trypsin and dispase; and
dispase and accutase.

23. The method according to claim 22, wherein said enzymatic composition is free from animal product and wherein said trypsin, dispase, collagenase, and accutase are recombinant trypsin, dispase, collagenase, and accutase, respectively.

24. The method according to claim 22 or claim 23, wherein said chicken cell preparation consists of more than 500 10⁶ cells extracted per embryo.

25. The method according to claim 22, wherein said chicken embryos are not dissected before said digestion.

26. The method according to claim 22, wherein said enzymatic composition is selected from the group consisting of:
trypsin added at a concentration equivalent to 30 mL/Embryo of TrypLE Select from Invitrogen Cat. No. 12563-029, 100 mg/Embryo of dispase, and 200 mg/Embryo of collagenase;
trypsin added at a concentration equivalent to 10 mL/Embryo of TrypLE Select from Invitrogen Cat. No. 12563-029, 100 mg/Embryo of dispase, and 200 mg/Embryo of collagenase;
trypsin added at a concentration equivalent to 15 mL/Embryo of TrypLE Select from Invitrogen Cat. No. 12563-029, 100 mg/Embryo of dispase, and accutase added at a concentration equivalent to 10 mL/Embryo of Accutase from Sigma Cat. No. A-6964;
trypsin added at a concentration equivalent to 15 mL/Embryo of TrypLE Select from Invitrogen Cat. No. 12563-029, 100 mg/Embryo of dispase, 200 mg/Embryo of collagenase, and accutase added at a concentration equivalent to 10 mL/Embryo of Accutase from Sigma Cat. No. A-6964;
trypsin added at a concentration equivalent to 30 mL/Embryo of TrypLE Select from Invitrogen Cat. No. 12563-029, 100 mg/Embryo of dispase, and accutase added at a concentration equivalent to 10 mL/Embryo of Accutase from Sigma Cat. No. A-6964;
trypsin added at a concentration equivalent to 10 mL/Embryo of TrypLE Select from Invitrogen Cat. No. 12563-029, 100 mg/Embryo of dispase, and 100 mg/Embryo of collagenase;
trypsin added at a concentration equivalent to 30 mL/Embryo of TrypLE Select from Invitrogen Cat. No. 12563-029, 100 mg/Embryo of dispase, 200 mg/Embryo of collagenase, and accutase added at a concentration equivalent to 10 mL/Embryo of Accutase from Sigma Cat. No. A-6964;
trypsin added at a concentration equivalent to 10 mL/Embryo of TrypLE Select from Invitrogen Cat. No. 12563-029, 100 mg/Embryo of dispase, and accutase added at a concentration equivalent to 10 mL/Embryo of Accutase from Sigma Cat. No. A-6964;
trypsin added at a concentration equivalent to 15 mL/Embryo of TrypLE Select from Invitrogen Cat. No. 12563-029, and 50 mg/Embryo of dispase;
trypsin added at a concentration equivalent to 30 mL/Embryo of TrypLE Select from Invitrogen Cat. No. 12563-029, and 100 mg/Embryo of dispase;
trypsin added at a concentration equivalent to 10 mL/Embryo of TrypLE Select from Invitrogen Cat. No. 12563-029, and 100 mg/Embryo of dispase;
100 mL/Embryo of dispase, and accutase 10 mL/Embryo; and
trypsin added at a concentration equivalent to 5 mL/Embryo of TrypLE Select from Invitrogen Cat. No. 12563-029, and 150 mg/Embryo of dispase.

27. The method according to claim 22, wherein said chicken cell preparation consists of isolated chicken cells.

28. The method according to claim 27, wherein said isolated chicken cells are chicken embryo fibroblasts (CEFs), chicken embryo kidney cells (CEKCs), or chicken embryo liver cells (CELCs).

29. The method according to claim 28, wherein said isolated chicken cells are chicken embryo fibroblasts (CEFs).

30. The method according to claim 22, wherein said chicken cell preparation consists of a mixture of chicken cells.

31. The method according to claim 30, wherein said mixture of chicken cells comprises CEFs, CEKCs, CELCs, cardiac cells, muscle cells, epithelial cells, blood cells, and/or endothelial cells.

32. A method for obtaining a chicken cell preparation by treating chicken embryos with an enzymatic composition, wherein said method comprises the steps of:
extracting embryos from opened eggs,
cutting the heads and feet, and
directly digesting the embryos, without a dissection step, by using an enzymatic composition according to claim 22, claim 23, or claim 26.

33. A method according to claim 32, wherein said digestion of said chicken embryos is performed under the following conditions:
a temperature of incubation between 35° C. and 39° C.; and
a duration of incubation between 1 and 3 hours.

34. The method according to claim 33, wherein the temperature of incubation is between 36° C. and 37° C.

35. The method according to claim 33, wherein the temperature of incubation is 36° C., 36.5° C., or 37° C.

36. The method according to claim 33, wherein the temperature of incubation is 37° C.

37. The method according to claim 33, wherein the duration of incubation is 2 hours.

38. The method according to claim 32, wherein said method further comprises one filtration step.

39. The method according to claim 38, wherein said filtration step is performed using a sieve made of inox.

40. The method according to claim 32, wherein said method further comprises one centrifugation step.

41. The method according to claim 21, wherein said chicken cell preparation is for use in the production of virus.

42. The method according to claim 41, wherein said virus is a poxvirus.
